(19) United States
(12) Patent Application Publication
(10) Pub. No.: US 2024/0054761 A1
(43) Pub. Date: Feb. 15, 2024

(54) IMAGING SYSTEM AND METHOD OF USE THEREOF

(71) Applicant: New York Stem Cell Foundation, Inc., NEW YORK, NY (US)

(72) Inventors: Brodie Fischbacher, New York, NY (US); Daniel John Paull, New York, NY (US); Zhongwei Wang, New York, NY (US)

(73) Assignees: New York Stem Cell Foundation, Inc., New York, NY (US); New York Stem Cell Foundation, Inc., New York, NY (US)

(21) Appl. No.: 17/766,439

(22) PCT Filed: Oct. 2, 2020
(86) PCT No.: PCT/US2020/054060

§ 371 Date: Apr. 4, 2022

Related U.S. Application Data
(60) Provisional application No. 63/051,310, filed on Jul. 13, 2020.
(60) Provisional application No. 62/971,017, filed on Feb. 6, 2020.
(60) Provisional application No. 62/910,951, filed on Oct. 4, 2019.

(51) Int. Cl. G06V 10/764; G06V 10/20; G06T 7/11; G06T 11/60; G06V 10/82

(52) U.S. Cl. CPC G06V 10/764; G06V 10/255; G06T 7/11; G06T 11/60; G06V 10/82; G06T 2207/10016; G06T 2210/22; G06T 2207/20084; G06T 2207/10056; G06T 2207/30072; G06V 2201/03

(57) ABSTRACT

The present disclosure provides a system and method for image analysis which utilize trained neural networks. The system and method are useful for generation and/or analysis of a variety of objects, such as biological cells to determine clonality.

### CROSS-REFERENCE TO RELATED APPLICATIONS

This application claims benefit of priority under 35 U.S.C. § 119(e) of U.S. Provisional Patent Application Ser. No. 62/910,951, filed Oct. 4, 2019; U.S. Provisional Patent Application Ser. No. 62/971,017, filed Feb. 6, 2020; and U.S. Provisional Patent Application Ser. No. 63/051,310, filed Jul. 13, 2020, the contents of which are incorporated herein by reference in their entireties.

### BACKGROUND OF THE INVENTION

### Field of the Invention

The invention relates generally to imaging, and more specifically to a system and method for generating an image of a target object and analyzing the target object within the generated image.

### Background Information

The isolation and subsequent expansion of a single cell derived from a cultured population establishes monoclonality and is frequently considered an essential step in developing high-quality cell lines. This procedure is intended to minimize or eliminate genomic and phenotypic heterogeneity in an attempt to maximize uniformity of cell lines. For instance, a newly genome-engineered cell population may comprise an admixture of cells with divergent alleles, zygosity and epigenetic characteristics. A homogenous cell line can thus only be reestablished by ensuring all cells in the population are descendent from a single ancestral cell which was isolated downstream of any event with a high proclivity to introduce variations. This step is referred to as monoclonalization.

An example of a cell culturing process in which monoclonalization is often considered critical is in that of human induced pluripotent stem cells (iPSCs). Due to the capacity for unlimited self-renewal and ability to differentiate via any lineage, this cell type offers immense promise for modelling disease states in vitro, enabling non-invasive genetic association studies, particularly as they relate to drug responses. Such efforts necessarily entail large, population-level cohorts. Cell-line derivation throughput is therefore the paramount limiting factor in unlocking the vast promise iPSC technology holds in relation to fields including functional genomics and precision medicine. The iPSC reprogramming process exerts a large amount of stress on cells, resulting in a population which is highly heterogeneous with regards to variables such as residual load of viral reprogramming vector and introduced chromosomal aberrations, eliciting the need to monoclonalize. Although fully automated methods for iPSC production have been described, the need for monoclonalization workflows in iPSC production remain, particularly when using viral vectors for iPSC vectors. As this step has historically incurred a critical bottleneck during automated and high-throughput derivation of iPSCs, this cell type is focused on as a case example for investigating monoclonalization methodologies.

Single-cell isolation is typically achieved via fluorescence-activated cell sorting (FACS), a form of flow cytometry. This process enables rapid sorting of individual cells, however there are a number means by which it can result in undesirable outcomes. Sorted cells may not survive, leaving an empty well; alternatively, faults in the sorting process may erroneously transfer more than one cell to the destination well, resulting in polyclonality. Further, for any given cell type, there may be variety of morphological or physiological changes that can occur during development that alter the quality of the cell line. In the case of stem cells (SCs), for instance, there are a number of known morphological markers which indicate loss of pluripotency, a common defect in newly reprogrammed iPSCs. As a result of these factors, the presence, clonality and quality of cell aggregations in putatively monoclonalized wells must be validated post-hoc.

At present, the only method for validating monoclonality is through manual inspection of microscopic imaging performed at regular intervals to track the growth of colonies after sorting. Doing so is highly time-consuming, with technicians often spending several hours per day classifying wells according to colony presence, clonality and morphology. More critically, however, the reliance on human judgement introduces key sources of bias and technical variability, particularly when such protocols are distributed among multiple investigators and research groups. As a result of this lack of standardization, monoclonalization protocols cannot be reliably upscaled without exacerbating the technical variability of cell lines. All of these factors make monoclonalization a highly desirable target for automation, which would enable colony selection protocols to be infinitely expanded and distributed at scale while minimizing technical variability.

Deep learning, based on the use of convolutional neural networks (CNNs), has enabled enormous advances in computer vision over the past several years and has become an invaluable tool in automating the analysis of biomedical images of various types. These techniques have already been applied to numerous processes in SC research, including for the automated inference of differentiation and prediction of function in iPSC-derived cell types. However, CNNs have never been employed in automatically identifying clonality during monoclonalization protocols for any cell type.

In domain-specific tasks, deep-learning models frequently match or surpass the image analyzing performance of human investigators. Dedicated neural network architectures exist for specific tasks such as image classification and segmentation. Specifically, detection networks, which are trained to detect and localize each instance of a given object class in images, clearly offer a promising opportunity for the automated verification of monoclonality, which ultimately relies on the counting of individual cells. Implementations of detection networks in other scientific endeavors have previously proven highly successful. These typically adhere to standardized procedures for training and inference, involving annotating images with object bounding boxes for training, followed by fitting the labelled data via defined network architectures such as region-based convolutional neural networks (RCNNs) and you only look once (YOLO).

A number of key nuances inherent to monoclonalization make the task resistant to automation through standardized, widely adopted deep learning practices. For instance, confirming a monoclonal well requires the enumeration of individual starting cells. These typically occupy <0.01% of the well's field of view and are frequently too small to be visible to human investigators without manually magnifying the image at the precise location of the cell. Grayscale imaging exacerbates this difficulty, typically exhibiting a large amount of noise. Debris particles very often appear subjectively indistinguishable from starting cells and investigators frequently rely upon information in later images, such as growth, to confirm whether a specific particle is a cell or an abiotic artefact.

Irrespective of the above, verifying clonality necessarily depends upon the interaction between images taken at different time points. For instance, enumerating individual cells in a day 0 image in order to validate that the sorting process was successful in isolating exactly 1 starting cell provides no information about the cell's subsequent survival, expansion or retention of desirable morphological traits. Conversely, validating that only a single colony is visible at time of inspection does not suffice to confirm monoclonality, given multiple starting cells may give rise to a single, polyclonal mass of cells which superficially resemble monoclonal colonies. In short, insofar as human investigators are able to assess, there are no cases in which a single image may contain all the information necessary to infer the clonality of a well. For this reason, it is not feasible to construct a conventional training set consisting simply of images and their corresponding semantic labels.

iPSCs are an attractive source of cells for therapeutic applications, medical research, pharmaceutical testing, and the like. However, there remains a longstanding need in the art for an automated system for rapidly producing and isolating reproducible iPSC cell lines under standard conditions in order to meet these and other needs.

### SUMMARY OF THE INVENTION

The present disclosure provides a system and method for image analysis based on a computational workflow, referred to herein as “Monoqlo” or the system of the invention, which integrates trained neural networks. While applicable to generation and analysis of many types of images, in one aspect, the system and method is useful for identifying and analyzing a biological cell, for example to determine a characteristic of the cell, such as a physical attribute, clonality, karyotype, phenotype, abnormality, disease state and the like.

Accordingly, in one embodiment, the invention provides an imaging system. The system includes an imaging device and a controller in operable connection to the imaging device, the controller being operable to generate images via the imaging device and analyze the generated images via a processor. In various aspects, the processor includes functionality to perform one or more of the following operations: i) generate a plurality of chronological images of an image area via the imaging device; ii) identify a target object within the image area of a most recent image of the plurality of chronological images; iii) generate a target object image area within the image area of the most recent image including the identified target object, the target object area having a perimeter within the image area of the most recent image; iv) use a prior image of the image area, and crop the prior image to generate a cropped image area sized to the perimeter of the target object image area; v) generate a location region of the cropped image area within the image area of the most recent image; and optionally vi) analyze the location region of the most recent image.

In another embodiment, the invention provides a method of performing image analysis. The method includes identifying and optionally analyzing a target object of an image using the system of the invention. In some aspects, analyzing the target object includes classifying the target object based on an attribute of the target object, such as a physical feature of the target object, including size and/or shape. In some aspects, the target object is a cell or cell colony and the physical attribute is a cell morphology feature, such as size and/or shape. In some aspects the attribute is a characteristic of the cell, such as clonality, karyotype, phenotype, abnormality and/or disease state.

In yet another embodiment, the invention provides an automated system for generating iPSCs or differentiated cells from iPSCs or SCs. The system includes: a) an induction unit for automated reprogramming of iPSCs or differentiation of SCs or iPSCs, the induction unit being operable to contact cells with reprogramming factors or differentiation factors; b) an imaging system operable to identify iPSCs or differentiated cells, wherein the imaging system comprises a non-transitory computer readable medium having instructions for identifying monoclonal or polyclonal cell populations; and optionally c) a sorting unit for isolating identified cells. In some aspects, the monoclonal or polyclonal cell populations are identified using one or more CNNs to process images taken by the imaging system of cells generated in a) which are cultured over a duration of time, thereby producing a set of images of the cells.

In another embodiment, the invention provides an automated method for generating iPSCs or differentiated cells from iPSCs or SCs. The method includes: a) generating an iPSC or differentiated cell from an SC or iPSC; b) identifying the iPSC or differentiated cell using an imaging system, wherein the imaging system comprises a non-transitory computer readable medium having instructions for identifying monoclonal or polyclonal cell populations; and optionally c) isolating the monoclonal or polyclonal cells via a sorting unit. In some aspects, the monoclonal or polyclonal cell populations are identified using one or more CNNs to process images taken by the imaging system of cells generated in a) which are cultured over a duration of time, thereby producing a set of images of the cells.

In another embodiment, the invention provides a non-transitory computer readable medium having instructions for identifying monoclonal or polyclonal cell populations. In various aspects, the non-transitory computer readable medium is electronically coupled to an imaging system.

In still another embodiment, the invention provides a method of determining the clonality of a cell population. The method includes: a) culturing a cell for a duration of time to generate a cell population; and b) analyzing the cell population over the duration of time utilizing an imaging system electronically coupled to a non-transitory computer readable medium of the present invention, thereby determining whether the cell population is monoclonal or polyclonal.

The invention also provides an automated system for analyzing a cell or cell population. The system includes: a) a cell culture unit for culturing a cell or cell population; b) an imaging system operable to analyze the cell or cell population, wherein the imaging system comprises a non-transitory computer readable medium having instructions for identifying morphological features of a cell or identifying monoclonal or polyclonal cell populations; and optionally c) a sorting unit for isolating a cell of interest from the cell culture unit.

In yet another embodiment, the invention provides an automated method for analyzing a cell or cell population. The method includes: a) culturing a cell or cell population; b) analyzing the cell or cell population using an imaging system, wherein the imaging system comprises a non-transitory computer readable medium having instructions for trained identifying morphological features of a cell or identifying monoclonal or polyclonal cell populations; and optionally c) isolating a cell of interest from the cultured cells.

In another embodiment, the invention provides a method that includes: a) culturing a cell in a sample well; and b) analyzing the cell using an imaging system of the invention, wherein the target object is the cell.

In still another embodiment, the invention provides an automated method for generating iPSCs or differentiated cells from iPSCs or SCs. The method includes: a) generating an iPSC or differentiated cell from an SC or iPSC; b) identifying the iPSC or differentiated cell using the imaging system of the invention, wherein the controller identifies monoclonal or polyclonal cell populations; and optionally c) isolating the monoclonal or polyclonal cells via a sorting unit.

In another embodiment, the invention provides a method of determining the clonality of a cell population. The method includes: a) culturing a cell for a duration of time to generate a cell population; and b) analyzing the cell population over the duration of time utilizing the imaging system of the invention, wherein the controller identifies monoclonal or polyclonal cell populations, thereby determining whether the cell population is monoclonal or polyclonal.

In yet another embodiment, the invention provides an automated system for analyzing a cell or cell population. The system includes: a) a cell culture unit for culturing a cell or cell population; b) the imaging system of the present invention, wherein the controller is operable to analyze the cell or cell population by identifying morphological features of a cell or identifying monoclonal or polyclonal cell populations; and optionally c) a sorting unit for isolating a cell of interest from the cell culture unit.

In another embodiment, the invention provides an automated method for analyzing a cell or cell population. The method includes: a) culturing a cell or cell population; b) analyzing the cell or cell population using the imaging system of the invention, wherein the controller is operable to analyze the cell or cell population by identifying morphological features of the cell or identifying monoclonal or polyclonal cell populations; and optionally c) isolating a cell of interest from the cultured cells.

### BRIEF DESCRIPTION OF THE FIGURES

FIG. 1A shows images of portions of four CNN modules utilized in one embodiment of the invention. Shown are simple schematic representations of two neural network architectures used for the tasks of detection and classification.

FIG. 1B shows images of portions of four CNN modules utilized in one embodiment of the invention. Shown are respective functionalities of each of 3 detection modules with representative target data and outputs.

FIG. 1C shows images of portions of four CNN modules utilized in one embodiment of the invention. Shown are examples of four target morphological classes used in training a morphological classification network of the invention.

FIG. 2 is a series of images illustrating an overview of the daily automation workflow which generates data for training and real-time use in one embodiment of the invention. Following cell deposition via FACs, cells are allowed to grow over N days, with well-level imaging occurring nightly. N represents a variable number dependent on cell growth rate and decisions on passage timing.

FIG. 3 is a schematic representing a broad overview of the design and algorithmic logic used in one embodiment of the invention. Arrows represent the processing order in the algorithm's reverse-chronological analysis, beginning with the most recent scan. If a colony is detected, the region around the colony is cropped in the previous day's scan and the image is passed to the local detection model. The process is repeated, progressively reducing the field of view being analyzed. If multiple colonies are detected in any scan, the well is declared polyclonal and no further scans are analyzed. Upon reaching the earliest “day 0” scan, the resulting image is passed to the local detection model. Based on the number of cells detected, a clonality for the well is finally declared.

FIG. 4A is an image showing results of validations generated with the present invention. Illustrated is well-level clonality identification performance of the framework of the invention on real-world production run data. Outer colors represent the ground-truthed clonality of the well, with color meanings indicated in legend; inner colors represent the clonality identified by the present invention, with dual-color wells thus indicating errors.

FIG. 4B is an image showing results of validations generated with the present invention. Illustrated is class-specific clonality identification performance of the present invention on manually curated, class-balanced test dataset.

FIG. 4C is an image showing results of validations generated with the present invention. Illustrate is a summary of clonality performance of the present invention, with analyses restricted to monoclonal, morphologically healthy wells that were selected for further passaging by biologists.

FIG. 5A is a graph providing a summary of classification model training and performance. Illustrated are training and validation accuracy trajectories of the classification CNN, plotted against epoch.

FIG. 5B is a graph providing a summary of classification model training and performance. Illustrated is a confusion matrix of fully trained classification CNN when tested on held-out validation is set.

FIG. 6 is a graph showing the relationship between width of colony bounding box predicted by a global detection model of the present invention and the true width measured by biologists with a scale bar image overlay

FIG. 7A is an image showing an example of abiotic artifacts causing false colony detections by the global detection model of the present invention. The image represents the image report generated by the present invention in full view.

FIG. 7B is an image showing an example of abiotic artifacts causing false colony detections by the global detection model of the present invention. The image represents the same image report show in FIG. 7A zoomed.

FIG. 8 is an image showing an example of overlapping reports of colonies by a local detection model of the present invention where only a single colony exists after ground-truthing.

FIG. 9 is an image showing an example of overlapping reports of colonies by a local detection model of the present invention where only a single colony exists after ground-truthing.

FIG. 10 is an image showing an example of overlapping reports of colonies by a local detection model of the present invention where only a single colony exists after ground-truthing.

FIG. 11 is an image illustrating the concept of “colony splitting”, where an apparent single colony is revealed, during reverse-chronological analysis, to have originated in multiple colonies which ultimately merged.

FIG. 12 is a series of graphs representative of a gating strategy employed during FACS-sort monoclonalization of iPSCs in one embodiment of the present invention.

### DETAILED DESCRIPTION OF THE INVENTION

The present invention is based on innovative system and method for image analysis. Before the present compositions and methods are described, it is to be understood that this invention is not limited to the particular system, method and/or experimental conditions described herein, as such systems, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the system” include one or more systems and references to “the method” include one or more methods, and/or steps of the type described herein which will become apparent to those persons skilled in the art upon reading this disclosure and so forth.

The present disclosure provides an imaging system and method for analysis of an imaged object which utilizes a computational workflow which integrates multiple CNNs. In some aspects, the present invention is based on a system and computational design which overcomes presently know difficulties by leveraging the chronological directionality inherent to the cell culturing process. The system and computational methodology described herein, termed Monoqlo, integrates multiple CNNs, each having its own “modular” functionality.

The present invention encompasses a highly scalable framework, capable of analyzing datasets numbering great than 1,000, 10,000, 50,000, 100,000, 500,000 or 1,000,000 images in a manageable timeframe of less than 10, 9, 8, 7, 6, 5, 4, 3, 2 or 1 hour. It will be appreciated that the functionality described herein may be applied to any number of conventional imagers. A discussed in detail in Example I, the work described herein demonstrates the first example of machine learning being applied to the identification of monoclonal cell lines from brightfield microscopy.

It will be understood that while the present disclosure illustrates imaging and analysis of biological cells, the system and method of the present invention are applicable to imaging any target object and subsequent analysis thereof.

Accordingly, in one embodiment, the invention provides an imaging system. The system includes an imaging device and a controller in operable connection to the imaging device, the controller being operable to generate images via the imaging device, and analyze the generated images via a processor. In various aspects, the processor includes functionality to perform one or more of the following operations: i) generate a plurality of chronological images of an image area via the imaging device; ii) identify a target object within the image area of a most recent image of the plurality of chronological images; iii) generate a target object image area within the image area of the most recent image including the identified target object, the target object area having a perimeter within the image area of the most recent image; iv) use a prior image of the image area, and crop the prior image to generate a cropped image area sized to the perimeter of the target object image area; v) generate a location region of the cropped image area within the image area of the most recent image; and optionally vi) analyze the location region of the most recent image.

The invention further provides a method of performing image analysis using the system of the invention. The method includes identifying and optionally analyzing a target object of an image using the system of the invention.

In some aspects, i)-vi) are iterated for each successive image of the plurality of chronological images. In some aspects, i)-vi) are iterated when only one target object is identified in the image area.

As discussed herein, the present invention is capable of analyzing image datasets of various sizes in a manageable timeframe. In some aspects, the dataset, for instance a plurality of chronological images, includes greater than 1, 10, 100, 1,000, 10,000, 100,000, 200,000, 300,000, 400,000, 500,000, 600,000, 700,000, 800,000, 900,000, 1,000,000 or more individual images.

As further discussed herein, the functionality described herein may be applied to any number of conventional imagers. As such, generation of an image for use with the present system and methodology may be accomplished in a variety of ways and analyzed and/or processed utilizing the functionality described herein. In some aspects, the system of the present invention includes one or more imaging devices operably coupled to the processor and/or other robotic platform components, such as a cell sorting unit, cell culturing unit, optical analyzer or assembly, cell reprogramming or differentiation unit, cryopreservation unit and the like. As used herein, an imaging device includes any device or detector capable of capturing an image including, but not limited to a camera, microscope, CCD camera, photodiode, photomultiplier tube, laser scanner and the like.

In various aspects, the system includes functionality to identify the target object in the location region of the most recent image and analyze the target object.

In some aspects, analyzing a target object includes classifying the target object based on an attribute of the target object. Such attributes may include a physical feature of the target object, such as size, shape and/or color.

In some aspects, the target object is a cell or cell colony and the attribute is a physical attribute including a cell morphology feature, such as size and/or shape. In some aspects, the attribute is a characteristic of the cell or cell colony, such as clonality, karyotype, phenotype, abnormality and/or disease state.

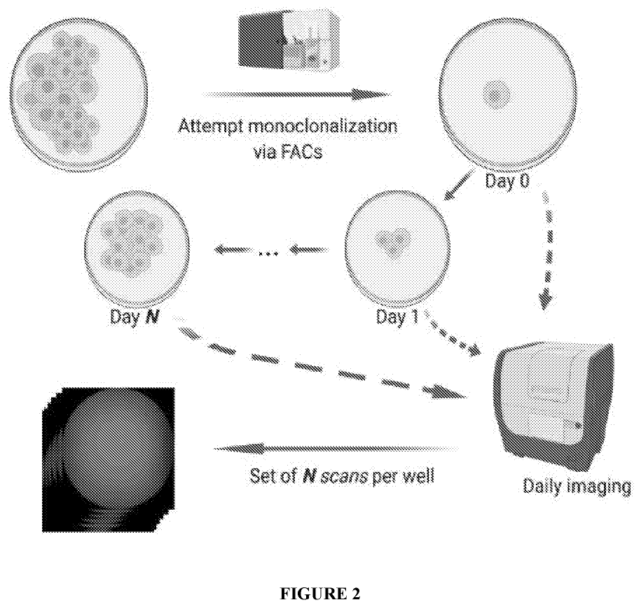

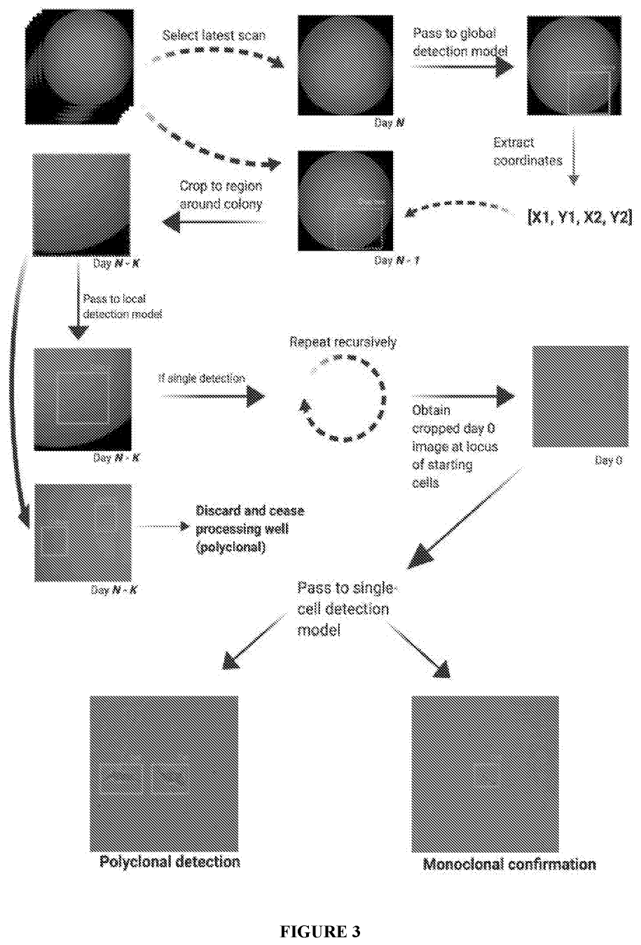

In various aspects, the system and method of the present invention integrate neural networks which may be trained for specific type of analysis and/or classification of a target object. The laboratory automation workflow which generates data is summarized in FIGS. 2 and 3, respectively. The algorithm processes images of an image area, typically including a target object, in a reversely chronological fashion. That is, for each image area, the algorithm begins by analyzing the most recently generated scan. In our case, this is an image that has been cropped only to remove the black borders of the image, preserving the entire field of the image area. These images are passed to the global detection model, the output of which is a coordinate vector demarcating the bounding boxes of any detected target objects.

The algorithm then expands these coordinates until each dimension of the bounding box is twice that of the predicted target object, loads the next most recent image for the image area and crops the image to the resulting region. Due to the preservation of physical positioning between scans, the earlier instantiation of the same target object is therefore approximately centered within the newly cropped image. This image is then passed to the local detection model, which reports the bounding box of the earlier target object, indicating its position within the original, uncropped image when summed with the cropping coordinates. The algorithm iterates this process recursively until the resultant most recent image is the earliest (“day 0”) scan.

In some aspects, a training set is stratified based on chronological timestamps, as well as magnification and crop level, and train separate neural networks, each having its own “modular” functionality. First, the term “global detection” is assigned to the task of detecting the presence or absence of a target object in an image area. Second, the task of detecting a target object in cropped image of various image areas at a variety of zoom magnifications is referred to as “local detection”. Third, the task of enumerating individual target objects in a fully magnified, cropped image was termed “single-cell detection”. It was sought to achieve all three of the aforementioned tasks through the use of the RetinaNet™ detection architecture with focal loss (Lin et al., In Proceedings of the IEEE international conference on computer vision; pp. 2980-2988 (2017)). Finally, a model was desired to categorize images cropped around colony regions into specific classes based on shape and/or size, such as morphological classes for cells, here referred to as “morphological classification”.

As illustrated in Example I, in various aspects, the system and methodology of the present invention identifies clonality of a cell or cell population, for example a monoclonal or polyclonal cell or cell population. Monoclonalization refers to the isolation and expansion of a single cell derived from a cultured population. This is typically done with the aim of minimizing a cell line's technical variability downstream of cell-altering events, such as reprogramming or gene editing, as well as for monoclonal antibody development. Without automated, standardized methods for assessing clonality post-hoc, methods involving monoclonalization cannot be reliably upscaled without exacerbating the technical variability of cell lines.

The present invention provides a deep learning workflow that automatically detects colony presence and identifies clonality from cellular imaging. As discussed in Example I, the workflow of the present invention integrates multiple convolutional neural networks and, critically, leverages the chronological directionality of the cell culturing process. The system and methodology described herein provides a fully scalable, highly interpretable framework, capable of analyzing industrial data volumes in under an hour using commodity hardware. In some aspects, the present invention standardizes the monoclonalization process, enabling colony selection protocols to be infinitely upscaled while minimizing technical variability.

As such, in another embodiment, the invention provides a non-transitory computer readable medium having instructions for identifying monoclonal or polyclonal cell populations. In various aspects, the non-transitory computer readable medium is electronically coupled to an imaging system.

In some aspects, the instructions provide for generating a set of images via the imaging system of cells being cultured over a duration of time, the set having a plurality of individual images. In some aspects, the individual images are taken in a chronological manner and assigned a chronological timestamp. In some aspects, the instructions further provide for processing the set of images in chronological order using one or more CNNs and categorizing the processed set of images based on morphological features of the cells and further classifying the cells as polyclonal or monoclonal based on the categorization.

In related embodiments, the invention further provides a method of determining the clonality of a cell population. The method includes: a) culturing a cell for a duration of time to generate a cell population; and b) analyzing the cell population over the duration of time utilizing an imaging system electronically coupled to a non-transitory computer readable medium of the present invention, thereby determining whether the cell population is monoclonal or polyclonal.

The invention also provides an automated system for analyzing a cell or cell population. The system includes: a) a cell culture unit for culturing a cell or cell population; b) an imaging system operable to analyze the cell or cell population, wherein the imaging system comprises a non-transitory computer readable medium having instructions for identifying morphological features of a cell or identifying monoclonal or polyclonal cell populations; and optionally c) a sorting unit for isolating a cell of interest from the cell culture unit. In some aspects, monoclonal and polyclonal cell populations are identified using one or more CNNs to process images taken by the imaging system of cells cultured in (a) which are cultured over a duration of time, thereby producing a chronological set of images of the cells over time. In some aspects, morphological features are identified and analyzed using one or more CNNs to process images taken by the imaging system of cells cultured in (a) which are cultured over a duration of time, thereby producing a chronological set of images of the cells over time.

In yet another embodiment, the invention provides an automated method for analyzing a cell or cell population. The method includes: a) culturing a cell or cell population; b) analyzing the cell or cell population using an imaging system, wherein the imaging system comprises a non-transitory computer readable medium having instructions for trained identifying morphological features of a cell or identifying monoclonal or polyclonal cell populations; and optionally c) isolating a cell of interest from the cultured cells. In some aspects, monoclonal and polyclonal cell populations are identified using one or more CNNs to process images taken by the imaging system of cells cultured in (a) which are cultured over a duration of time, thereby producing a chronological set of images of the cells over time. In some aspects, morphological features are identified and analyzed using one or more CNNs to process images taken by the imaging system of cells cultured in (a) which are cultured over a duration of time, thereby producing a chronological set of images of the cells over time.

As is clear from the disclosure, the present invention is useful in generating iPSCs or differentiated cells in which identification and/or classification of monoclonal cell populations is desired. As such, in one embodiment, the invention provides an automated system for generating iPSCs or differentiated cells from iPSCs or SCs. The system includes: a) an induction unit for automated reprogramming of iPSCs or differentiation of SCs or iPSCs, the induction unit being operable to contact cells with reprogramming factors or differentiation factors; b) an imaging system operable to identify iPSCs or differentiated cells, wherein the imaging system comprises a non-transitory computer readable medium having instructions for identifying monoclonal or polyclonal cell populations; and optionally c) a sorting unit for isolating identified cells.

In another embodiment, the invention provides an automated method for generating iPSCs or differentiated cells from iPSCs or SCs. The method includes: a) generating an iPSC or differentiated cell from an SC or iPSC; b) identifying the iPSC or differentiated cell using an imaging system, wherein the imaging system comprises a non-transitory computer readable medium having instructions for identifying monoclonal or polyclonal cell populations; and optionally c) isolating the monoclonal or polyclonal cells via a sorting unit.

In some aspects, the monoclonal or polyclonal cell populations are identified using one or more CNNs to process images taken by the imaging system of cells generated in a) which are cultured over a duration of time, thereby producing a set of images of the cells.

In some aspects, sorting of cells is accomplished by a cell dispensing or sorting technology, which may optionally include flow cytometry. For example, cells may be sorted using single cell sorting, fluorescence-activated cell sorting (FACS), and/or magnetic activated cell sorting (MACS).

As used herein “adult” means post-fetal, e.g., an organism from the neonate stage through the end of life, and includes, for example, cells obtained from delivered placenta tissue, amniotic fluid and/or cord blood.

As used herein, the term “adult differentiated cell” encompasses a wide range of differentiated cell types obtained from an adult organism, that are amenable to producing iPSCs using the instantly described automation system. Preferably, the adult differentiated cell is a “fibroblast.” Fibroblasts, also referred to as “fibrocytes” in their less active form, are derived from mesenchyme. Their function includes secreting the precursors of extracellular matrix components including, e.g., collagen. Histologically, fibroblasts are highly branched cells, but fibrocytes are generally smaller and are often described as spindle-shaped. Fibroblasts and fibrocytes derived from any tissue may be employed as a starting material for the automated workflow system on the invention.

As used herein, the term, “induced pluripotent stem cells” or, iPSCs, means that the stem cells are produced from differentiated adult cells that have been induced or changed, e.g., reprogrammed into cells capable of differentiating into tissues of all three germ or dermal layers: mesoderm, endoderm, and ectoderm. The iPSCs produced do not refer to cells as they are found in nature.

The terms “stem cell” or “undifferentiated cell” as used herein, refer to a cell in an undifferentiated or partially differentiated state that has the property of self-renewal and has the developmental potential to differentiate into multiple cell types, without a specific implied meaning regarding developmental potential (e.g., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. While each of these multipotent cells may be considered stem cells, the range of cell types each such stem cell can give rise to, e.g., their developmental potential, can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

The term “differentiated cell” encompasses any somatic cell that is not, in its native form, pluripotent, as that term is defined herein. Thus, the term a “differentiated cell” also encompasses cells that are partially differentiated, such as multipotent cells, or cells that are stable, non-pluripotent partially reprogrammed, or partially differentiated cells, generated using any of the compositions and methods described herein. In some embodiments, a differentiated cell is a cell that is a stable intermediate cell, such as a non-pluripotent, partially reprogrammed cell. The transition of a differentiated cell (including stable, non-pluripotent partially reprogrammed cell intermediates) to pluripotency requires a reprogramming stimulus beyond the stimuli that lead to partial loss of differentiated character upon placement in culture. Reprogrammed and, in some embodiments, partially reprogrammed cells, also have the characteristic of having the capacity to undergo extended passaging without loss of growth potential, relative to parental cells having lower developmental potential, which generally have capacity for only a limited number of divisions in culture. In some embodiments, the term “differentiated cell” also refers to a cell of a more specialized cell type (e.g., decreased developmental potential) derived from a cell of a less specialized cell type (e.g., increased developmental potential) (e.g., from an undifferentiated cell or a reprogrammed cell) where the cell has undergone a cellular differentiation process.

The term “reprogramming” as used herein refers to a process that reverses the developmental potential of a cell or population of cells (e.g., a somatic cell). Stated another way, reprogramming refers to a process of driving a cell to a state with higher developmental potential, e.g., backwards to a less differentiated state. The cell to be reprogrammed can be either partially or terminally differentiated prior to reprogramming. In some embodiments of the aspects described herein, reprogramming encompasses a complete or partial reversion of the differentiation state, e.g., an increase in the developmental potential of a cell, to that of a cell having a pluripotent state. In some embodiments, reprogramming encompasses driving a somatic cell to a pluripotent state, such that the cell has the developmental potential of an embryonic stem cell, e.g., an embryonic stem cell phenotype. In some embodiments, reprogramming also encompasses a partial reversion of the differentiation state or a partial increase of the developmental potential of a cell, such as a somatic cell or a unipotent cell, to a multipotent state. Reprogramming also encompasses partial reversion of the differentiation state of a cell to a state that renders the cell more susceptible to complete reprogramming to a pluripotent state when subjected to additional manipulations, such as those described herein. Such manipulations can result in endogenous expression of particular genes by the cells, or by the progeny of the cells, the expression of which contributes to or maintains the reprogramming. In certain embodiments, reprogramming of a cell using the synthetic, modified RNAs and methods thereof described herein causes the cell to assume a multipotent state (e.g., is a multipotent cell). In some embodiments, reprogramming of a cell (e.g., a somatic cell) using the synthetic, modified RNAs and methods thereof described herein causes the cell to assume a pluripotent-like state or an embryonic stem cell phenotype. The resulting cells are referred to herein as “reprogrammed cells,” “somatic pluripotent cells,” and “RNA-induced somatic pluripotent cells.” The term “partially reprogrammed somatic cell” as referred to herein refers to a cell which has been reprogrammed from a cell with lower developmental potential by the methods as disclosed herein, such that the partially reprogrammed cell has not been completely reprogrammed to a pluripotent state but rather to a non-pluripotent, stable intermediate state. Such a partially reprogrammed cell can have a developmental potential lower that a pluripotent cell, but higher than a multipotent cell, as those terms are defined herein. A partially reprogrammed cell can, for example, differentiate into one or two of the three germ layers, but cannot differentiate into all three of the germ layers.

The term a “reprogramming factor,” as used herein, refers to a developmental potential altering factor, as that term is defined herein, such as a gene, protein, RNA, DNA, or small molecule, the expression of which contributes to the reprogramming of a cell, e.g., a somatic cell, to a less differentiated or undifferentiated state, e.g., to a cell of a pluripotent state or partially pluripotent state. A reprogramming factor can be, for example, transcription factors that can reprogram cells to a pluripotent state, such as SOX2, OCT3/4, KLF4, NANOG, LIN-28, c-MYC, and the like, including as any gene, protein, RNA or small molecule, that can substitute for one or more of these in a method of reprogramming cells in vitro. In some embodiments, exogenous expression of a reprogramming factor, using the synthetic modified RNAs and methods thereof described herein, induces endogenous expression of one or more reprogramming factors, such that exogenous expression of one or more reprogramming factors is no longer required for stable maintenance of the cell in the reprogrammed or partially reprogrammed state.

As used herein, the term “differentiation factor” refers to a developmental potential altering factor, as that term is defined herein, such as a protein, RNA, or small molecule, which induces a cell to differentiate to a desired cell-type, e.g., a differentiation factor reduces the developmental potential of a cell. In some embodiments, a differentiation factor can be a cell-type specific polypeptide, however this is not required. Differentiation to a specific cell type can require simultaneous and/or successive expression of more than one differentiation factor. In some aspects described herein, the developmental potential of a cell or population of cells is first increased via reprogramming or partial reprogramming using synthetic, modified RNAs, as described herein, and then the cell or progeny cells thereof produced by such reprogramming are induced to undergo differentiation by contacting with, or introducing, one or more synthetic, modified RNAs encoding differentiation factors, such that the cell or progeny cells thereof have decreased developmental potential.

In the context of cell ontogeny, the term “differentiate”, or “differentiating” is a relative term that refers to a developmental process by which a cell has progressed further down a developmental pathway than its immediate precursor cell. Thus in some embodiments, a reprogrammed cell as the term is defined herein, can differentiate to a lineage-restricted precursor cell (such as a mesodermal stem cell), which in turn can differentiate into other types of precursor cells further down the pathway (such as a tissue specific precursor, for example, a cardiomyocyte precursor), and then to an end-stage differentiated cell, which plays a characteristic role in a certain tissue type, and may or may not retain the capacity to proliferate further.

The present invention includes a system and processor for performing steps of the disclosed method and is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions.

A method for image analysis according to various aspects of the present invention may be implemented in any suitable manner, for example using a computer program operating on the computer system. An exemplary analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing information may be implemented in a single device or implemented in a plurality of devices. The software may be accessible via a network such that storage and processing of information takes place remotely with respect to users. The analysis system according to various aspects of the present invention and its various elements provide functions and operations to facilitate image analysis, such as data gathering, processing, analysis, classification and/or reporting. For example, in the present embodiment, the computer system executes the computer program, which may receive, store, search, analyze, classify and/or report information relating to an image, cell or cell population. The computer program may include multiple modules performing various functions or operations, such as a processing module for processing raw data and generating supplemental data and an analysis module for analyzing raw data and supplemental data to generate quantitative assessments of a target object.

As used in this specification and the appended claims, the singular forms “a,” “an,” and “the” include plural referents, unless the context clearly dictates otherwise. The terms “a” (or “an”), as well as the terms “one or more,” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology, P-S. Juo, (2d ed. 2002) can provide one of skill with general definitions of some terms used herein.

Units, prefixes, and symbols are denoted in their Systeme International de Unites (SI) accepted form. Numeric ranges are inclusive of the numbers defining the range. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example I

Modular Deep Learning Enables Automated Identification of Monoclonal Cell Lines.

The present Example describes a system and computational method which leverages the chronological directionality inherent to the cell culturing process. The computational workflow, integrates multiple CNNs, each having its own “modular” functionality.

The system and methodology of the invention provides a highly scalable framework, which were capable of analyzing datasets numbering in the tens of thousands of images in under an hour. Through the combination of automated stem cell culture and deep learning, this work demonstrates the first example of machine learning being applied to the identification of monoclonal cell lines from brightfield microscopy.

Methods

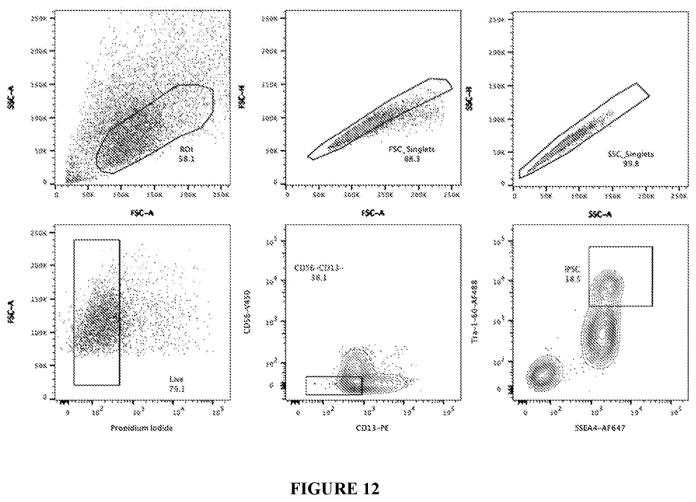

Monoclonalization of hiPSCs. Destination plates (PerkinElmer #6005182) were pre-coated with 17 ug Geltrex™ LDEV-Free, hESC-Qualified, Reduced Growth Factor Basement Membrane Matrix™ (ThermoFisher #A1413302) diluted in 50 uL DMEM/F12 (ThermoFisher #A1413302) for 1 hr in a 37 C incubator. Following incubation, 150 uL of d0 media 1×DMEM/F12, 1.5×PSC Freedom™ Supplement, (ThermoFisher #A27336SA), 1.5×Antibiotic/Antimycotic (ThermoFisher #15240062) and 15% CloneR™ (Stemcell Technologies #05888) was added to the 50 uL of Geltrex™+DMEM/F12 present in the well and incubated for 1 hr in a 37 C incubator. hiPSC colonies maintained on Geltrex™ in Freedom™ PSC media (FRD1) (both ThermoFisher) were dissociated with Accutase™ (ThermoFisher #A1110501) for 5-10 min at 37 C. Accutase™ was quenched with Sort™ Buffer (MACS Buffer Miltenyi, containing 10% CloneR™) and the cell suspension pelleted by centrifugation at 130 RCF. Cells were stained with antibodies: SSEA4-647: 1:100; BD #560219, Tra-1-60-488: 1:100; BD #560173, CD56-V450: 1:100; BD #560360, CD13-PE: 1:100; BD #555394 before being rinsed with a second centrifugation and resuspended in Sort™ Buffer+Propidium Iodide (PI, 1:5000, ThermoFisher #P3566). Cells were then sorted using a FACSARIA-IIu™ Cell Sorter (BD Biosciences) into the pre-prepared destination plates using a 100 μm ceramic nozzle with a sheath pressure of 23 psi. The flow cytometry gating strategy employed is summarized in FIG. 12. For samples sorted using the WOLFSorter™, the Sort™ Buffer was supplemented with SYTOX AADvanced™ Ready Flow™ Reagent (ThermoFisher #R37173) instead of PI.

Image acquisition and labelling. All images were sourced from repositories of historical data from the monoclonalization step employed during the iPSC production process of the NYSCF Global Stem Cell Array®. These images, previously used for manually verifying clonality, are generated automatically once per 24-hour period from seeding until plates are disposed of. All scans, which were generated by Nexcelom™ Celigo cytometers, are brightfield images at a resolution of 1 μm per pixel, providing an image dimension of 7544×7544 pixels after stitching from 16 field individual fields. The inventors annotated a total of 3,139 images with bounding boxes and object classes. An additional 2,224 unannotated images of empty wells were included in the training set as background-only images. During preliminary investigations, doing so was determined to be pivotal in reducing the rate of false detections. All annotations were generated in Pascal VOC format using the LabelImg™ software (Tzutalin, 2015). The dataset was augmented by applying random flip and rotation transforms to the images (as per e.g., Perez & Wang, 2017). The morphological criteria required for categorizing each object class were designated by PhD-level biologists specializing in iPSC culture. Annotations were made by technicians of PhD-, MS- and BS-level, with all annotations being independently corroborated by an additional investigator.

Training of machine learning models. RetinaNet™ detection models were trained using a Keras RetinaNet™ implementation (github.com/fizyr/keras-retinanet) using a ResNet50™ convolutional backbone (He et al., In Proceedings of the IEEE conference on computer vision and pattern recognition; pp. 770-778 (2016)) without pretrained weights. Preprocessing involved subtracting ImageNet™ means from images and normalizing pixel intensity values to the range between 0 and 1. The inventors also implemented a hand-crafted algorithm for cropping the thick black borders around the well from the image which removes the outermost line on each edge of the image and repeats until the maximum, raw pixel intensity value for the given line exceeds 70. Each CNN model was trained for 60 epochs, with weights being saved after each epoch, allowing the checkpoint with the smallest validation loss to be selected as the final model for use in the Monoqlo framework.

Results

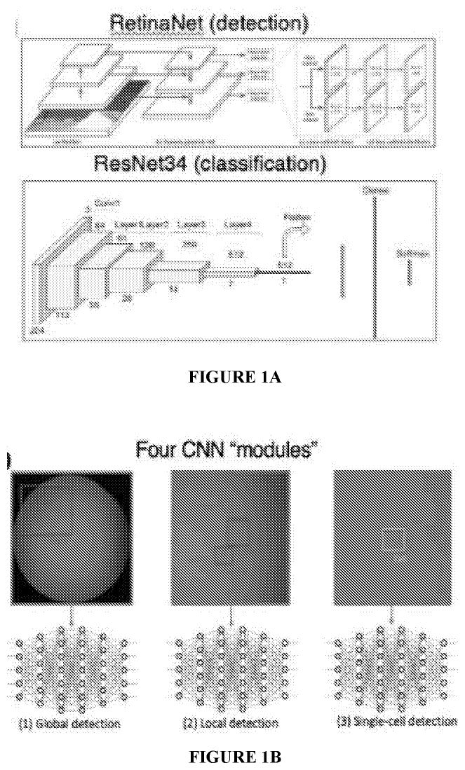

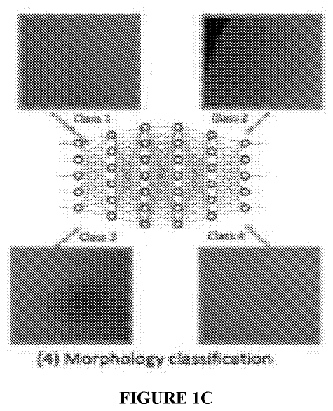

Neural network modularity. The task of automatically assigning clonality into four distinct deep-learning-enabled functionalities was modularized (FIG. 1). The decision to modularize was based upon empirical inferences made during preliminary investigations. Namely, consistent with the principles of transfer learning, it was initially suspected that a CNN's feature-extracting capacity would be best optimized by consolidating all image types into a single training set. However, it was found that networks trained in this manner performed poorly, often failing to distinguish between object classes. In particular, they often reported object types that could not feasibly occur in the image in question, for instance detecting fully developed colonies in images generated immediately after seeding. This indicated that a single model would not perform well across the diversity of image magnifications and object classes employed during monoclonalization.

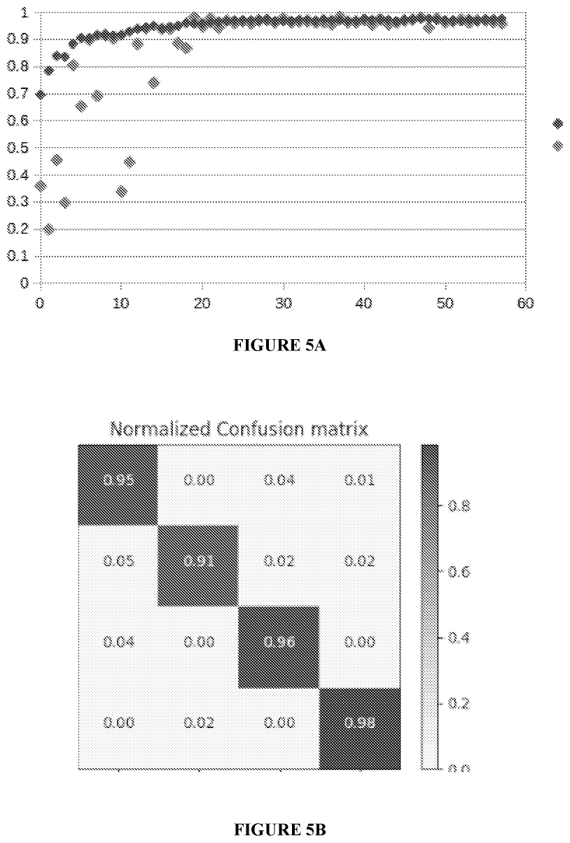

Instead, the training set was stratified based on chronological timestamps, as well as magnification and crop level, and train four separate neural networks, each having its own “modular” functionality. First, the term “global detection” is assigned to the task of detecting the presence or absence of colonies in a full-well image. Second, the task of detecting colonies in cropped images of various well regions at a variety of zoom magnifications is referred to as “local detection”. Third, the task of enumerating individual cells in a fully magnified, cropped image was termed “single-cell detection”. It was sought to achieve all three of the aforementioned tasks through the use of the RetinaNet™ detection architecture with focal loss (Lin et al., In Proceedings of the IEEE international conference on computer vision; pp. 2980-2988 (2017)). Finally, in the only entirely classification-based task in this effort, a model was desired to categorize images cropped around colony regions into morphological classes, here referred to as “morphological classification” (summarized in FIGS. 5A-5B). Modularizing in this manner enabled the inventors to capitalize on the temporal directionality of the cell culturing process; for instance, restricting detectable object classes to those that may realistically exist in an image based on its scan date.

Workflow design overview. A computational workflow was designed, termed Monoqlo, which integrates each of the trained neural networks. The laboratory automation workflow which generates data for use with Monoqlo and the design of Monoqlo itself are summarized in FIGS. 2 and 3, respectively. The algorithm processes images on a per-well basis in a reversely chronological fashion. That is, for each physical well, the algorithm begins by analyzing the most recently generated scan. In our case, this is an image that has been cropped only to remove the black borders of the image, preserving the entire field of the physical well. These images are passed to the global detection model, the output of which is a coordinate vector demarcating the bounding boxes of any detected colonies.

The algorithm then expands these coordinates until each dimension of the bounding box is twice that of the predicted colony, loads the next most recent image for the same well and crops the image to the resulting region. Due to the preservation of plate orientation and physical positioning between scans, the earlier instantiation of the same colony is therefore approximately centered within the newly cropped image. This image is then passed to the local detection model, which reports the bounding box of the earlier colony, indicating its position within the original, uncropped image when summed with the cropping coordinates. The algorithm iterates this process recursively until the resultant most recent image is the earliest (“day 0”) scan, generated within hours of sorting. It was found that this incremental, iterative processing aspect of the workflow, as well as the expansion of the crop box dimensions, to be essential, as there are invariably small deviations from precise concentricity with each day due to non-radial growth and minor positional shifts between scans. Over periods of several days of imaging, these deviations sum to substantial offsets. As such, simply cropping and magnifying at the exact center of a late-stage colony will rarely yield a field of view in which the starting cell or cells are situated.

Aside from counting individual starting cells, polyclonality can often be inferred if two or more clearly distinct cell masses are observed, which are assumed to have originated from two or more cells from the same FACS sort. If either the global or local detection models reports a colony count of >1 at any point during the process of iterating backwards chronologically, the algorithm accordingly declares the well to be polyclonal and ceases processing any further images for that well. Alternatively, if the workflow continues to detect exactly one colony until reaching the day-zero scan, the resulting image will be magnified and cropped exactly around the ancestral cell or cells. This image can then be passed to the single-cell detection model, providing a count of the number of starting cells. On this basis, the well may then finally be declared either monoclonal or polyclonal.

Chronological processing logic enables optimization. In this case, any given monoclonalization “run” typically comprises between 300 and 900 plate wells and 2-6 runs are typically active at any one time. With per-well scans occurring daily for between 12 and 30 days the mean volume for each run at time of processing by the algorithm is therefore approximately 30,000 images. Rather than pertaining to images, however, the target labels in the case of monoclonalization correspond to individual wells. For this reason, a “well knockout” approach is used in which detection by the workflow of any one of a number of exclusion criteria causes the algorithm to eliminate the entire well from the workflow and ignore all subsequent scans for that well. For instance, if no objects are detected in the most recent scan, then the well is reported empty at time of analysis and its antecedent characteristics are considered irrelevant. During testing, Monoqlo was executed on 8 plates of 96 wells. The mean number of empty wells per plate at time of processing was found to be 73, ranging from 41 to 92. Thus, in cases where Monoqlo is applied to, for instance, 8 plates at day 15 of the monoclonalization process, the well knockout approach alleviates the need for processing of approximately 8,760 of a total of 11,400 images (76.8%) on the basis of emptiness alone. Any well found to be polyclonal at any stage of analysis is also excluded from further processing. In the same test run, a mean of 11 polyclonal wells per plate were found, with polyclonality being declared after a mean of 5.73 images having been processed. During real time deployment, the exclusion criteria was further extended to eliminate wells which are found to exhibit the morphological markers of differentiation. Due to the enormity of the datasets which require daily analysis, our knockout approach provides a vast improvement to compute time.

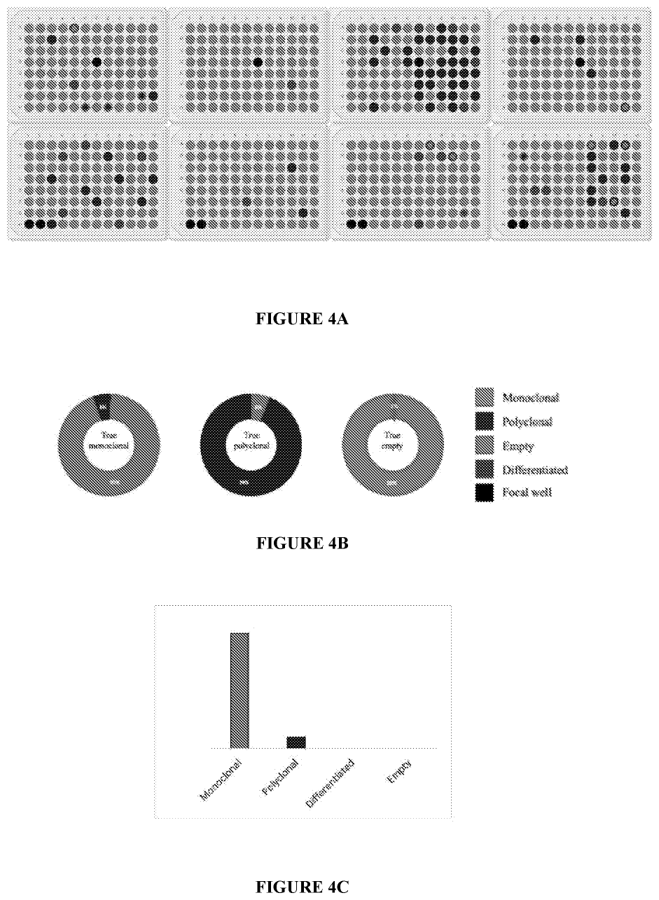

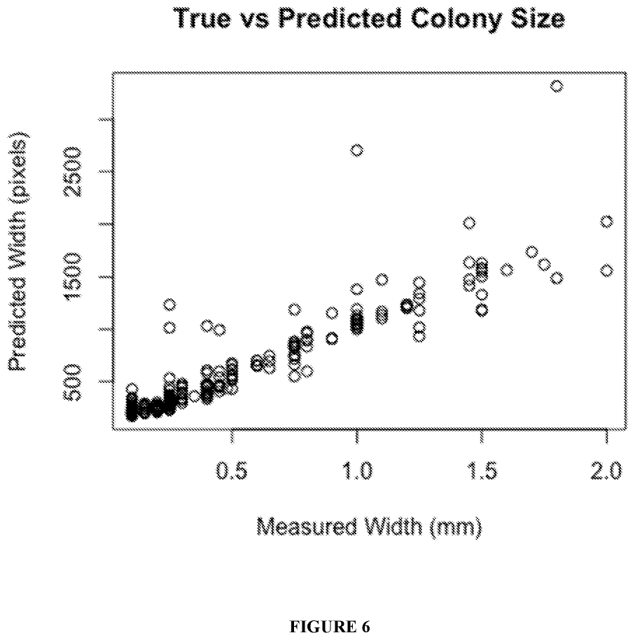

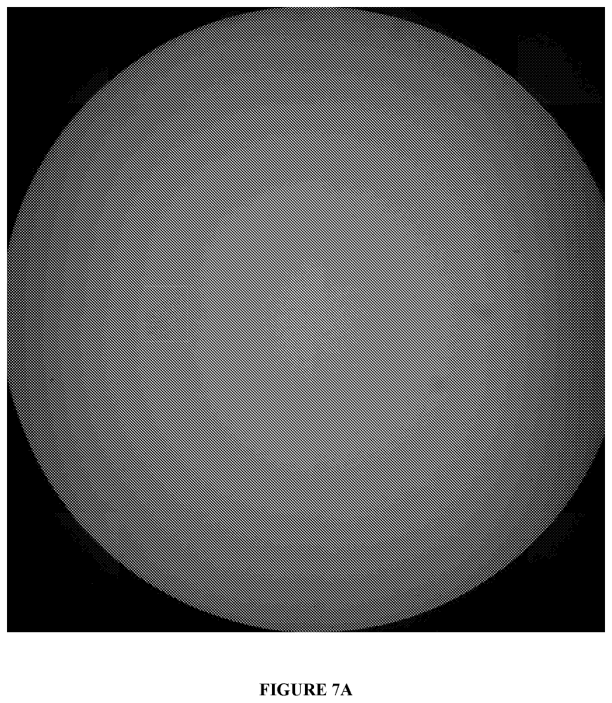

Neural networks learn to detect colonies and classify morphology. The inventors began by evaluating learning trajectories and benchmarking the prediction performance of each CNN in its respective task. In the case of object detection networks, our initial metrics for assessment were the change in value of the loss function when tested on a held-out validation dataset representing 20% of our total image set. While training such networks, precise accuracy metrics are not automatically generated by the learning algorithms, since the model may correctly detect an object without the labeled and predicted bounding box coordinates matching exactly. As an alternative, their performance was manually evaluated by visually comparing labels and predictions in validation images with their respective bounding boxes drawn. From these comparisons, detection performance was quantified according to two metrics: 1) percentage of labelled objects which were correctly predicted and classified, and 2) number false positives, in which the model detected an object where none was present, as a ratio to the total number of images analyzed. Results of our model validations are summarized in FIG. 4. Finally, true colony width, as measured by biologists using an image scale bar, was highly predicted by bounding box X dimension (Pearson's r(266)=0.917, p<2.2e-16) (FIG. 6).

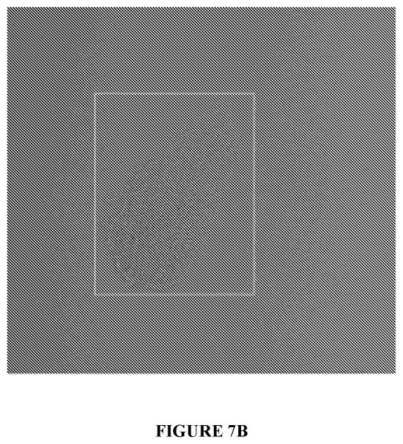

Deep-learning workflow with modularization identifies clonality. The efficacy of Monoqlo as a unified, modular workflow, was benchmarked first by testing its accuracy on a manually curated, class-balanced validation set, and subsequently by evaluating its clonality identification performance (irrespective of morphology) post-hoc on a raw, unfiltered dataset from real-world monoclonalization runs. The curated test set included 100 wells from each of three classes; empty, monoclonal and polyclonal; randomly selected from historical records of manually classified wells. The imaging date at which processing was initiated for each well was randomly generated from the range of days 8-18. The real-world scenario validation was performed on a monoclonalization run (DMR0001) which comprised 768 wells in total, spanning a time frame of 19 days, thus yielding a data volume of 18,240 images. Manual image review found 561 of these wells to be empty; that is, they contained no indication of living cells, irrespective of remnants of dead colonies, abiotic debris and other artefacts. Monoqlo correctly eliminated 556 (99.1%) of these wells. The remaining 5 empty wells were reported as monoclonal, seemingly resulting in false positives on the part of the global detection model due to unidentified abiotic artefacts (FIG. 7) with a superficially similar appearance to that of a cell colony. Accordingly, Monoqlo identified 194 non-empty wells. This included 115 monoclonal declarations, of which 2 and 5 wells were found to have ground truth classifications of “polyclonal” and “empty”, respectively; the remaining 108 wells (93.9%) being concordant with the ground truth. Finally, 61 wells were reported as being polyclonal, of which 57 (93.4%) were confirmed by ground truthing and 4 were found to be monoclonal. Results of both validations are summarized in FIG. 5.

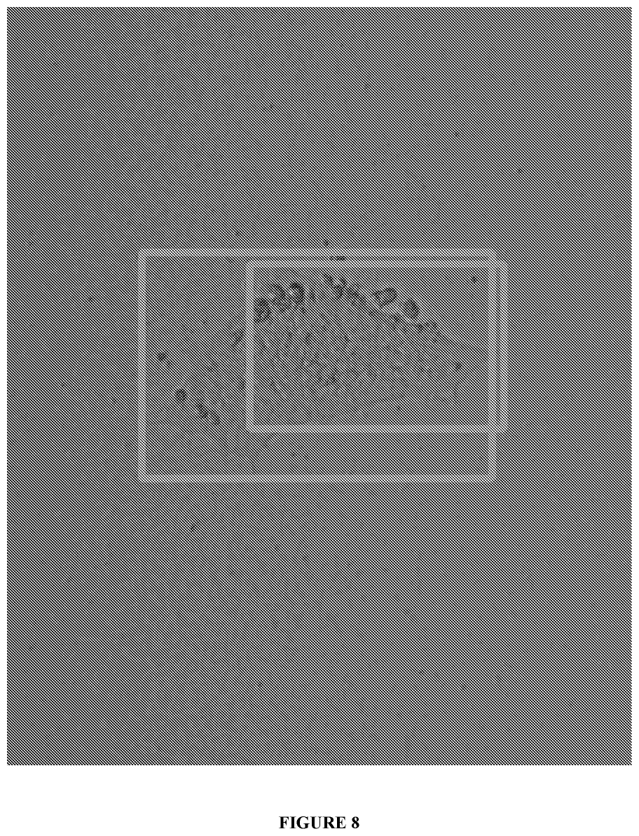

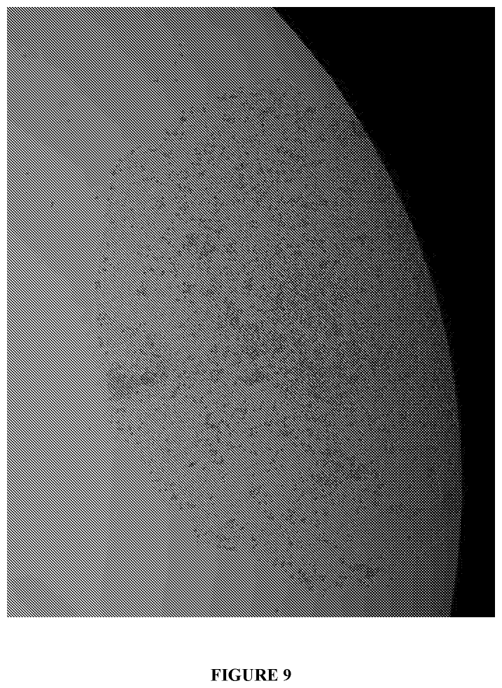

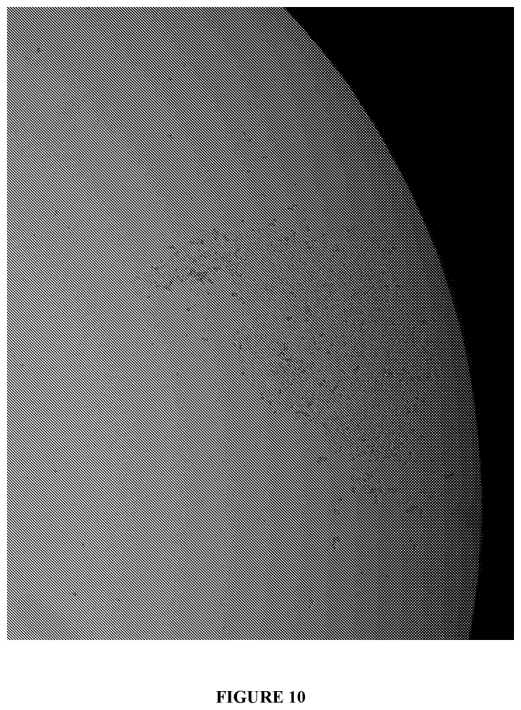

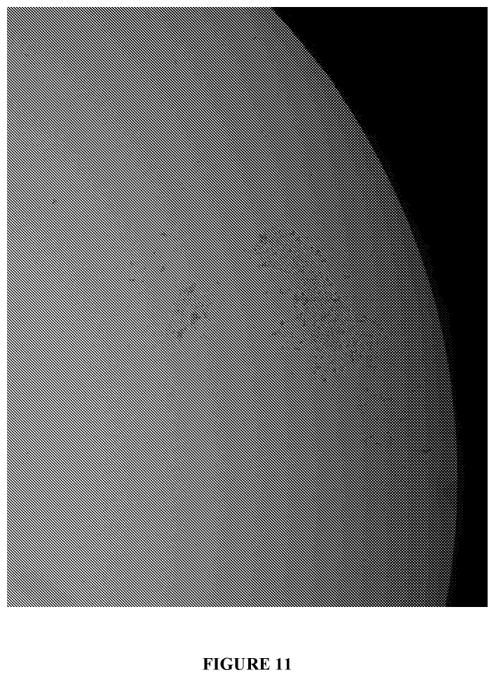

Hand-crafted programmatic solutions improve deep learning workflows. A number of circumstances were identified in which shortcomings of our trained CNNs, which would otherwise have led to erroneous results, could be robustly corrected for using simple programmatic logic. Perhaps most prominently, it was found that detection CNNs tended to often report multiple, overlapping colonies in image regions where only a single colony existed in the ground truth (FIG. 8). It was possible to partially mitigate this by adjusting the size and distribution of the anchor boxes. However, doing so is laborious, can only be done prior to training a model, and provides only an incomplete solution. Instead, the algorithm combines any overlapping boxes and considers the resulting box as a single object. In the case of colony detection, this never results in loss of polyclonal identifications. To illustrate, consider the concept of “colony splitting” which occurs due to Monoqlo's reversely chronological approach. Colonies which overlap one another at day N are spatially isolated at day N-K and have grown into a combined mass at day N+K where K is a variable amount of time dependent on growth rates and original separation distance (FIGS. 9-11). Thus, overlapping object detections can safely be considered by our algorithm as a single object which, if representing multiple colonies, will later be detected as entirely isolated from one another in earlier images and thus declared polyclonal.

Discussion

This work represents the first successful attempt to automate the identification of clonality using a deep learning object detection approach. It is expected that this has the potential to remove a critical restriction on scalability in a number of cell culturing domains. This includes the present case of iPSC derivation, where monoclonalization is considered essential for two reasons. First, in cases of viral reprogramming, there is a large amount of cell-to-cell variance in residual load of the Sendai viral vector used to deliver transcription factors to the inner cell during reprogramming. Second, the reprogramming process often leads to severe chromosomal abnormalities, presumably due to stress-induced mitotic disruptions. Both of these factors cause profound phenotypic variation, resulting in unpredictable, highly heterogeneous cell lines, eliciting the need for monoclonalization, which has historically incurred a bottleneck during iPSC production. It was suggested that the physical monoclonalization process could exert further physiological stress on cells, however single cell cloning remains critical in a number of use cases. Given the extent to which cohort size dictates the viability of population studies, the removal of this bottleneck, as demonstrated in the present work, represents a major step in fully unlocking the immense research potential of iPSCs.

Perhaps more significantly, in addition to initial derivation, huge efforts are being made towards optimizing CRISPR-Cas9 editing efficiency and other forms of genome engineering in iPSCs, which holds enormous potential in regard to functionally annotating gene variants, disease modelling and validating polymorphisms identified in genetic association studies. Due to the genomic heterogeneity the editing process introduces, newly edited populations must be monoclonalized to ensure that all cells carry the same genotypes. While the inventors have focused on iPSCs in the present study, the same holds true for gene editing in all cell types. The genome engineering pipeline is therefore viewed to be another critical case in which the Monoqlo framework alleviates a major bottleneck in disease research and therapeutic development.

It was suspected that the algorithm could be adapted to any cell type, provided the cells are capable of being imaged and form discrete clonal masses. As an important example, antibody development is one of the most common use cases for monoclonalization, due to the epitope specificity of monoclonal antibodies. Many of the most frequently used cell types in antibody development have been successfully detected in microscopy imaging with CNNs. As monoclonal antibodies form the central component of many drug discovery efforts, the Monoqlo framework may have the potential to offer a valuable tool to the pharmaceutical industry at large.

The present study adds to previous instances of deep learning applications in iPSC process automation. In particular, there is a great deal of interest in optimizing CNNs for use with brightfield microscopy in an effort to alleviate the need for immunostaining and fluorescence microscopy imaging, which comes at much larger costs to financial investment and investigation time. For instance, a previous attempt successfully trained deep learning models to predict fluorescent labels from brightfield images alone. This work further demonstrates the predictive power of deep learning in various analysis tasks using simple microscopy images without the requirement of fluorescent labelling.

It was shown that standard CNN architectures such as Resnet50 ™ may be trained to distinguish differentiated and undifferentiated stem cells in culture, even at early onset. The classification CNN of the present invention differs from those previously described in that the training classes are stratified to a greater extent, as opposed to a binary “differentiated versus undifferentiated” approach. Doing so served to increase the robustness of our algorithm when applied in real-world cell culturing scenarios, in which there is a high degree variability in iPSC colony morphology due to factors other than pluripotency status. Additionally, the network is trained on images cropped around distinct, singular colonies as opposed to field-of-view images containing numerous, randomly seeded cell aggregations. In this sense, our training data are more akin to that employed in which a vector-based CNN is used to distinguish “healthy” from “unhealthy” colonies. However, this approach requires significant hand-crafted preprocessing steps and, critically, requires manual cropping of exact colony regions, restricting its utility in real-world automation scenarios. By using the classification network in conjunction with colony detection models, the inventors automate the segmentation step, enabling fully autonomous deployment in laboratory automation scenario.

Shortcomings for the approach are noted. For instance, in cases where two or more starting cells are displayed precisely adjoining one another in the earliest available scan, the well's clonality status must be considered ambiguous. This is because it cannot be determined whether the cells were sorted independently from the source plate or if a single cell was successfully sorted in isolation and subsequently divided. Notably, however, there is a time lag between seeding and attachment of the cell to the substrate during which the cell cannot be imaged. For this reason, the timing window of the first scan is critical. Certain other efforts have attempted to address this ambiguity through fluorescence microscopy applied to nuclear-stained images, which allows nuclear segmentation and helps to resolve the spatial distribution of individual cells. However, this does not entirely eliminate ambiguity since physically adjacent cells, even if clearly distinct, could certainly still have a polyclonal origin. It was suggested that there are a limited number of feasible approaches to handling this ambiguity. Investigators may wish to simply assume any well containing multiple cells at time of earliest scan is polyclonal. Otherwise, it is suspected that the ambiguity can only be resolved by generating images taken within minutes of seeding. Due to the time lag that occurs before cells can attach, however, optical focusing issues will be inevitable. Thus, starting cells are likely to be invisible at times, making it impossible to reliably verify monoclonality.

In the present study, the 100% detection rate for colonies of sufficient size for passaging suggests Monoqlo's suitability for deployment as a dependable, fully autonomous system.

It is expected that Monoqlo could help facilitate investigations in a number of key questions which remain to be answered with regard to the predictive potential of deep neural networks in iPSC research. A number of studies have demonstrated that deep learning approaches can sometimes discriminate between biological groups in images where a morphological phenotype was not previously known to exist; or was suspected to exist but was not visible to even a trained human investigator. For instance, it was shown that CNNs can predict factors such as cardiovascular disease risk, gender and smoking status from individual retinal images, none of which was previously thought to manifest morphologically in the retina. Further, in the case of iPSCs, deep neural networks have been successfully trained to predict donor identity from imaging of clinical-grade iPSC-derived retinal pigment epithelium. With these discoveries in mind, the likely existence of thus far unidentified predictive markers in iPSC colony morphology is suggested. For instance, it may be possible to predict with better-than-random accuracy at an early stage whether a presently undifferentiated colony will spontaneously differentiate. Successfully training such a model would confer enormous benefit to iPSC derivation, given the substantial costs associated with continuing to culture cells which may ultimately become unusable. Other candidate targets for CNN classification- or regression-based prediction include Sendai virus load, future QC pass/fail status and relative differentiation affinity for specific germ layers.

Training such models will invariably require large training volumes. The Monoqlo framework allows colonies to be algorithmically segmented and cropped from raw datasets, in addition to automatically filtering out images of empty wells which typically represent the vast majority of images. In many cases, investigators may also be able to label images in batch on the basis of the classification they assign to the most recent image of a given colony or well. Applying the classification network, which identifies differentiation, allows Monoqlo to retroactively assign labels such as “will differentiate” or “won't differentiate” to earlier instantiations of the colony. This may mitigate the need for extensively laborious, manual reviews and labelling of unfiltered image sets, enabling partially or fully autonomous generation of large training volumes for future models. As such, our algorithm provides an invaluable tool for generating custom datasets for future investigations of the utility of deep learning in iPSC research.

In summary, a framework has been demonstrated in which deep learning algorithms with a modular design can automate the verification of monoclonality in brightfield microscopy, requiring relatively little labelling. The functionality of the workflow was further expanded to classification of colony morphology, demonstrating the potential for autonomous monitoring of monoclonal cell line development and clonal selection in automation workflows. Monoqlo represents a crucial step in enabling widespread distribution of high-throughput cell line production and editing workflows. This may eliminate a critical bottleneck in the specific case of iPSC derivation and genome editing, moving current technology closer to the goal of unrestricted upscaling and distribution of pluripotent stem cells for biomedical research applications. Finally, in contrast to depending solely on machine learning models to contend with all aspects of a given task, this work is viewed as a useful example to highlight the benefit of combining the now well-recognized, immense capabilities of convolutional neural networks with human-designed algorithmic solutions.

Although the invention has been described with reference to the above examples, it will be understood that modifications and variations are encompassed within the spirit and scope of the invention. Accordingly, the invention is limited only by the following claims.

What is claimed is:

1. An imaging system comprising:
a) an imaging device; and
b) a controller in operable connection to the imaging device, the controller being operable to generate images via the imaging device, and analyze the generated images via a processor, wherein the processor includes functionality to:
i) generate a plurality of chronological images of an image area via the imaging device;
ii) identify a target object within the image area of a most recent image of the plurality of chronological images;
iii) generate a target object image area within the image area of the most recent image including the identified target object, the target object area having a perimeter within the image area of the most recent image;
iv) use a prior image of the image area, and crop the prior image to generate a cropped image area sized to the perimeter of the target object image area;
v) generate a location region of the cropped image area within the image area of the most recent image; and
vi) analyze the location region of the most recent image.

2. The system of claim 1, wherein i)-vi) are iterated for each successive image of the plurality of chronological images.

3. The system of claim 2, wherein the plurality of chronological images comprises greater than 10, 100, 1,000, 10,000, 100,000 or more individual images.

4. The system of claim 2, wherein i)-vi) are iterated when only one target object is identified in the image area.

5. The system of claim 1, further comprising identifying the target object in the location region of the most recent image.

6. The system of claim 5, further comprising analyzing the target object.

7. The system of claim 6, wherein analyzing the target object comprises classifying the target object based on an attribute of the target object.

8. The system of claim 7, wherein the attribute is a physical feature of the target object.

9. The system of claim 8, wherein the physical feature is size or shape.

10. The system of claim 1, wherein i)-vi) are performed via one or more convolutional neural networks (CNNs).

11. The system of claim 1, wherein the target object is a cell or cell colony.

12. The system of claim 11, wherein cells of the cell colony are monoclonal.

13. A method of performing image analysis comprising identifying and optionally analyzing a target object of an image using the system of any of claims 1-12.

14. The method of claim 13, wherein analyzing the target object comprises classifying the target object based on an attribute of the target object.

15. The method of claim 14, wherein the attribute is a physical feature of the target object.

16. The method of claim 15, wherein the physical feature is size or shape.

17. The method of claim 15, wherein the target object is a cell or cell colony and the physical attribute is a cell morphology feature.

18. An automated system for generating induced pluripotent stem cells (iPSCs) or differentiated cells from iPSCs or stem cells (SCs) comprising:
a) an induction unit for automated reprogramming of iPSCs or differentiation of SCs or iPSCs, the induction unit being operable to contact cells with reprogramming factors or differentiation factors;
b) an imaging system operable to identify iPSCs or differentiated cells, wherein the imaging system comprises a non-transitory computer readable medium having instructions for identifying monoclonal or polyclonal cell populations; and optionally
c) a sorting unit for isolating identified cells.

19. The system of claim 18, wherein monoclonal or polyclonal cell populations are identified using one or more CNNs to process images taken by the imaging system of cells generated in (a) which are cultured over a duration of time, thereby producing a set of images of the cells.

20. The system of claim 19, wherein the images are processed in a chronological manner.

21. The system of claim 20, wherein each of the images is assigned a chorological timestamp.

22. The system of claim 21, further comprising categorizing the set of images based on morphological features of the cells.

23. The system of claim 22, further comprising classifying the cells as polyclonal or monoclonal based on the categorization.

24. The system of claim 24, further comprising isolating a cell classified as monoclonal via the sorting unit.

25. The system of claim 24, wherein sorting is optionally performed via a cell dispensing technology.

26. The system of claim 19, wherein the set of images comprises greater than 1, 10, 100, 1,000, 10,000, 15,000, 20,000, 25,000, 30,000, 50,000 or 100,000 images.

27. An automated method for generating iPSCs or differentiated cells from iPSCs or SCs, comprising:
a) generating an iPSC or differentiated cell from an SC or iPSC;
b) identifying the iPSC or differentiated cell using an imaging system, wherein the imaging system comprises a non-transitory computer readable medium having instructions for identifying monoclonal or polyclonal cell populations; and optionally
c) isolating the monoclonal or polyclonal cells via a sorting unit.

28. The method of claim 27, wherein monoclonal or polyclonal cell populations are identified using one or more CNNs to process images taken by the imaging system of cells generated in (a) which are cultured over a duration of time, thereby producing a set of images of the cells.

29. The method of claim 28, wherein the images are processed in a chronological manner.

30. The method of claim 29, wherein each of the images is assigned a chorological timestamp.

31. The method of claim 30, further comprising categorizing the set of images based on morphological features of the cells.

32. The method of claim 31, further comprising classifying the cells as polyclonal or monoclonal based on the categorization.

33. The method of claim 32, further comprising isolating a cell classified as monoclonal via the sorting unit.

34. The method of claim 27, wherein sorting is optionally performed via a cell dispensing technology.

35. The method of claim 27, wherein the set of images comprises greater than 10,000, 15,000, 20,000, 25,000 or 30,000 images.

36. A non-transitory computer readable medium having instructions for identifying monoclonal or polyclonal cell populations.

37. The non-transitory computer readable medium of claim 36, wherein the medium is electronically coupled to an imaging system.

38. The non-transitory computer readable medium of claim 37, wherein the instructions provide for generating a set of images via the imaging system of cells being cultured over a duration of time, the set having a plurality of individual images.

39. The non-transitory computer readable medium of claim 38, wherein the set of images comprises greater than 10,000, 15,000, 20,000, 25,000 or 30,000 images.

40. The non-transitory computer readable medium of claim 38, wherein the individual images are taken in a chronological manner and assigned a chronological timestamp.

41. The non-transitory computer readable medium of claim 40, wherein the instructions provide for processing the set of images in chronological order using one or more CNNs.

42. The non-transitory computer readable medium of claim 41, wherein the instructions provide for categorizing the processed set of images based on morphological features of the cells.

43. The non-transitory computer readable medium of claim 42, wherein the instructions provide for classifying the cells as polyclonal or monoclonal based on the categorization.

44. The non-transitory computer readable medium of claim 43, wherein the instructions provide for isolating a cell classified as monoclonal or polyclonal.

45. A method of determining the clonality of a cell population comprising:
a) culturing a cell for a duration of time to generate a cell population; and
b) analyzing the cell population over the duration of time utilizing an imaging system electronically coupled to a non-transitory computer readable medium of any of claims 36-44, thereby determining whether the cell population is monoclonal or polyclonal.

46. An automated system for analyzing a cell or cell population comprising:
a) a cell culture unit for culturing a cell or cell population;
b) an imaging system operable to analyze the cell or cell population, wherein the imaging system comprises a non-transitory computer readable medium having instructions for identifying morphological features of a cell or identifying monoclonal or polyclonal cell populations; and optionally
c) a sorting unit for isolating a cell of interest from the cell culture unit.

47. The system of claim 46, wherein monoclonal or polyclonal cell populations are identified using one or more CNNs to process images taken by the imaging system of cells cultured in (a) which are cultured over a duration of time, thereby producing a chronological set of images of the cells over time.

48. The system of claim 46, wherein morphological features are identified and analyzed using one or more CNNs to process images taken by the imaging system of cells cultured in (a) which are cultured over a duration of time, thereby producing a chronological set of images of the cells over time.

49. An automated method for analyzing a cell or cell population comprising:
a) culturing a cell or cell population;
b) analyzing the cell or cell population using an imaging system, wherein the imaging system comprises a non-transitory computer readable medium having instructions for trained identifying morphological features of a cell or identifying monoclonal or polyclonal cell populations; and optionally
c) isolating a cell of interest from the cultured cells.

50. The method of claim 49, wherein monoclonal or polyclonal cell populations are identified using one or more CNNs to process images taken by the imaging system of cells cultured in (a) which are cultured over a duration of time, thereby producing a chronological set of images of the cells over time.

51. The method of claim 49, wherein morphological features are identified and analyzed using one or more CNNs to process images taken by the imaging system of cells cultured in (a) which are cultured over a duration of time, thereby producing a chronological set of images of the cells over time.

52. A method comprising:
a) culturing a cell in a sample well; and
b) analyzing the cell using an imaging system of any of claims 1-12, wherein the target object is the cell.

53. An automated method for generating iPSCs or differentiated cells from iPSCs or SCs, comprising:
a) generating an iPSC or differentiated cell from an SC or iPSC;
b) identifying the iPSC or differentiated cell using the imaging system of any of claims 1-12, wherein the controller identifies monoclonal or polyclonal cell populations; and optionally
c) isolating the monoclonal or polyclonal cells via a sorting unit.

54. A method of determining the clonality of a cell population comprising:
a) culturing a cell for a duration of time to generate a cell population; and
b) analyzing the cell population over the duration of time utilizing the imaging system of any of claims 1-12, wherein the controller identifies monoclonal or polyclonal cell populations, thereby determining whether the cell population is monoclonal or polyclonal.

55. An automated system for analyzing a cell or cell population comprising:
a) a cell culture unit for culturing a cell or cell population;
b) the imaging system of any of claims 1-12, wherein the controller is operable to analyze the cell or cell population by identifying morphological features of a cell or identifying monoclonal or polyclonal cell populations; and optionally
c) a sorting unit for isolating a cell of interest from the cell culture unit.

56. An automated method for analyzing a cell or cell population comprising:
a) culturing a cell or cell population;
b) analyzing the cell or cell population using the imaging system of any of claims 1-12, wherein the controller is operable to analyze the cell or cell population by identifying morphological features of the cell or identifying monoclonal or polyclonal cell populations; and optionally
c) isolating a cell of interest from the cultured cells.

57. The system of claim 12, wherein the system further comprises a cell isolation module for isolating monoclonal cells.

58. The system of claim 57, wherein the system further comprises a protein isolation module for isolating protein produced by the monoclonal cells.

59. The system of claim 25, wherein the cell dispensing technology is FACS.

60. The method of claim 34, wherein the cell dispensing technology is FACS.

61. An automated system for analyzing a cell or cell population comprising:
a) a cell culture unit for culturing a cell or cell population;
b) an imaging system operable to analyze the cell or cell population, wherein the imaging system comprises a non-transitory computer readable medium having instructions for identifying a characteristic of the cell or cell population; and optionally
c) a sorting unit for isolating a cell of interest from the cell culture unit.

62. The system of claim 61, wherein the characteristic is identified using one or more CNNs to process images taken by the imaging system of cells cultured in (a) which are cultured over a duration of time, thereby producing a chronological set of images of the cells over time.

63. The system of any of claim 61 or 62, wherein the characteristic is a morphological feature, clonality, karyotype, phenotype, abnormality and/or disease state.

64. An automated method for analyzing a cell or cell population comprising:
a) culturing a cell or cell population;
b) analyzing the cell or cell population using an imaging system, wherein the imaging system comprises a non-transitory computer readable medium having instructions for trained identifying of a characteristic of a cell or cell population; and optionally
c) isolating a cell of interest from the cultured cells.

65. The method of claim 64, wherein characteristic is identified using one or more CNNs to process images taken by the imaging system of cells cultured in (a) which are cultured over a duration of time, thereby producing a chronological set of images of the cells over time.

66. The method of any of claim 64 or 65, wherein the characteristic is a morphological feature, clonality, karyotype, phenotype, abnormality and/or disease state.

67. A method comprising:
a) culturing a cell in a sample well; and
b) analyzing the cell using an imaging system of any of claims 1-12, wherein the target object is the cell.

68. The method of claim 68, wherein analyzing comprises determining a characteristic of the cell identified using one or more CNNs to process images taken by the imaging system of the cell cultured over a duration of time, thereby producing a chronological set of images of the cell over time.

69. The method of any of claim 67 or 68, wherein the characteristic is a morphological feature, clonality, karyotype, phenotype, abnormality and/or disease state.
